(19) United States
(12) Patent Application Publication
(10) Pub. No.: US 2006/0093555 A1
(43) Pub. Date: May 4, 2006

(54) Imaging inflammatory conditions using superparamagnetic iron oxide agents

(76) Inventors: Andrew Soliz Torres, Clifton Park, NY (US); Brian Christopher Bales, Niskayuna, NY (US); Peter John Bonitatebus JR., Saratoga Springs, NY (US); Amit Mohan Kulkarni, Clifton Park, NY (US); William Thomas Dixon, Clifton Park, NY (US); Lisa Anne Schoonmaker, Watervliet, NY (US); Patrick Roland Lucien Malenfant, Clifton Park, NY (US); Anton Beletskii, Niskayuna, NY (US); Matthew Sam Morrison, Amersham (GB)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/263,714

(22) Filed: Nov. 1, 2005

Related U.S. Application Data
(63) Continuation-in-part of application No. 10/818,235, filed on Apr. 2, 2004.

(51) Int. Cl. A61K 49/10

(52) U.S. Cl. 424/9.36; 977/930

(57) ABSTRACT

The present invention is directed to the field of magnetic resonance imaging (MRI) using superparamagnetic iron oxide (SPIO) agents. In particular, the present invention is directed to cationic, nonagglomerated, nontoxic SPIO agents, methods for imaging conditions associated with inflammatory responses using the disclosed SPIO agents, and methods for managing inflammatory conditions using the disclosed SPIO agents.

### CROSS-REFERENCE TO RELATED APPLICATIONS

This application claims benefit of priority to U.S. Ser. No. 10/818,235, entitled “Nanoparticles with Inorganic Core and Methods of Using Them,” filed on Apr. 2, 2004 and U.S. Ser. No. 10/10/989,632, entitled “Cationic Nanoparticle Having an Inorganic Core,” filed on Nov. 15, 2004, which are both incorporated herein by reference.

### FIELD OF THE INVENTION

The present invention is directed to the field of magnetic resonance imaging (MRI) using superparamagnetic iron oxide (SPIO) agents. In particular, the present invention is directed to charged, nonagglomerated, nontoxic SPIO agents, methods for imaging conditions associated with inflammatory responses using the SPIO agents of the invention, methods for managing conditions associated with inflammatory responses using the SPIO agents of the invention.

### BACKGROUND OF THE INVENTION

Inflammatory responses within blood vasculature and tissue result in the recruitment of immune response cells to the site of disease or injury. Immune response cells (e.g., macrophage cells, dendritic cells (DCs), polynuclear monocytes (PNMs), eosinophils, neutrophils, and T cells) are know to participate in immune responses that cause inflammatory diseases, including diseases of the central nervous system, vascular disease, and autoimmune disease. The compositions and methods of the present invention harness inflammatory response cells to deliver positively charged SPIO agents to the inflammatory foci and facilitate imaging of the inflamed tissue.

In magnetic resonance imaging (MRI), the image of an organ or tissue is obtained by placing a subject in a strong external magnetic field and observing the response (typically the response of the hydrogen nuclei of water) present in the subject's organs or tissues after excitation by a radio frequency magnetic field. The proton relaxation times, termed as T1 (longitudinal relaxation time) and T2 (transverse relaxation time) depend on the chemical and physical environment of the organ or tissue water protons. T1 and T2 vary from tissue to tissue and strongly affect image intensity. To generate a magnetic resonance image with good contrast, the T1, T2, and/or T2* of the tissue to be imaged must be different from the background tissue. One way of improving contrast of MR images is to use a MRI contrast agent.

Existing MRI contrast agents, such as paramagnetic metal complexes or superparamagnetic iron oxides, have several disadvantages. For example, although existing paramagnetic contrast agents can reduce T1 and thereby improve contrast, the paramagnetic contrast agents suffer from various disadvantages, such as adverse clinical reactions, short blood circulation times, and potential toxicity. Many paramagnetic metal complexes are hypertonic and often result in adverse clinical reactions upon injection.

Known SPIO agents consist of iron oxide cores stabilized by biocompatible coatings such as dextran, starch, or carbohydrate. Typically, the iron oxide core diameter ranges from about 3 to about 10 nm and the diameter of the core and coating combined ranges from about 10 to about 100 nm. Known SPIO agents, such as Feridex® and Resovist®, are negatively charged and have a short blood residence time (human blood half-life of less than 1 hour) precluding them from accessing tissue with slow uptake. Hence, agents with a short blood residence time are ill suited for imaging such tissue and subendothelial spaces, for example, the intima of blood vessels. Existing superparamagnetic particle contrast agents also suffer from various disadvantages, such as wide size distribution, agglomeration, instability, and toxicity.

Combidex®, with a dextran coating and a diameter of 15-30 nm, has been evaluated for magnetic resonance imaging in a variety of animal disease models as well as in humans. Due to its small size, Combidex® has a long blood residence time (human blood half-life between 24-36 hours). However, Combidex® is not readily taken up by inflammatory response cells, requiring doses that are substantially greater than the currently approved human dose for iron up to 2.6 mg Fe/kg body weight.

Needs remain for SPIO agents of appropriate solubility, biocompatibility, size, and coating characteristics that are capable of being efficiently internalized by inflammatory response cells and trafficked to the site of inflammation for use in imaging inflamed tissue.

The SPIOs described herein are substantially nontoxic, non-agglomerated, water soluble imaging agents that are optimally coated and sized for efficient internalization by inflammatory response cells such as, for example, macrophages, monocytes and polymorphonuclear cells (PNMs) that home to the site of inflammatory diseases. Accordingly, the contrast agents and methods of the present invention are useful for the diagnosis and management of inflammatory diseases, including for example, autoimmune disease, heart disease, circulatory disorders, lung disease, brain disease, and/or cancer.

### SUMMARY

The advantages and features of the invention disclosed herein will be made more apparent from the description, drawings, and claims that follow.

The present inventors have determined that uptake of SPIO agents by inflammatory response cells is improved by coating small SPIO molecules with charged shells that are soluble, polydispersed, and cationic. The disclosed SPIO agents demonstrate low toxicity, and are well tolerated by inflammatory response cells. Furthermore, the disclosed SPIO agents and methods of using them provide enhanced images of conditions associated with inflammatory response cells infiltration and accumulation. Such conditions may include autoimmune conditions, vascular conditions, neurological conditions, or combinations thereof.

In one embodiment, a method of imaging an inflammatory condition in a mammal comprising introducing into the mammal a SPIO agent including a superparamagnetic core and a cationic coating into inflammatory cells. The methods of imaging an inflammatory condition may be performed in vivo or ex vivo. In some embodiments, the mammal is a human.

In other embodiments, the disclosed methods further comprise treating the mammal to decrease inflammation before, after, or before and after imaging the inflammatory condition. In other additional embodiments of the disclosed methods further comprise using the imaging results to manage the inflammatory condition.

SPIO agents useful in the disclosed methods include a superparamagnetic core comprising divalent metal ion(s). The divalent metal irons may include iron, manganese, nickel, cobalt, magnesium, or a combination thereof. The size of the core may be about 2 nm to about 200 nm, about 2 nm to about 100 nm, or about 5 nm to about 9 nm. In other embodiments, the size of core is about 9 nm, about 7 nm, or about 5 nm.

The disclosed SPIO agents are substantially coated with a “shell” that is taken up by inflammatory response cells. The shells may comprise PEG, PEI, or combinations thereof. In embodiments where the shell comprises PEG, the PEG coating comprises: PEG-silane, PEG-dendron; PEG-dendron-silane, or combinations thereof. In embodiments using dendritic structures, the dendritic structure may include a single (G0) or multiple branches. In other embodiments, the PEG has a molecular weight between about 350 Da to about 5000 Da, about 550 Da to about 1000 Da. In other embodiments the coating comprises the shell coating S101, S104 (depicted in FIG. 1) or a combination thereof.

In another aspect, the size of the core and shell combined, measured by D_{H} may be about 3 nm to about 50 nm. In other embodiments, the D_{H} of the core and coating is about 17 nm. In still other embodiments, the SPIO agent is less than about 15% polydispersed

The disclosed methods of imaging inflammatory conditions may use cationic SPIO agents. In some embodiments the zeta potential of the agent is greater than about 0 and less than about +60 mV or about +20 mV to about +40 mV. In other embodiments, the zeta potential of the agent is about +40 mV.

In some embodiments, The R1 relaxivity of the SPIO agents useful in the disclosed methods may be greater than about 4 mM⁻¹s⁻¹ or greater than about 20 mM⁻¹s⁻¹. Furthermore, the R2/R1 ratio of the agent may be greater than about 2.

In another aspect, the SPIO agents may be dispersed in a biocompatible solution with a pH of about 6 to about 8. In other embodiments, the SPIO agent useful in the disclosed methods is dispersed in a biocompatible solution with a pH of about 7 to about 7.5. In yet other embodiments, the SPIO agent is dispersed in a biocompatible solution with a pH of about 7.4.

In another aspect the SPIO agents used in the disclosed methods of imaging inflammatory conditions has a blood half-life of the agent is about 30 minutes to about 48 hours or about 30 minutes to about 2 hours.

In the disclosed methods of imaging inflammatory conditions the SPIO agent may introduced to the mammal topically, intravascularly, intramuscularly, or interstitially. In some embodiments, about 0.1 mg Fe/kg to about 50 mg Fe/kg of agent is administered to the mammal. In other embodiments, about 0.2 mg Fe/kg to about 2.5 mg Fe/kg of agent is administered to the mammal.

These and other advantages and features of the invention disclosed herein, will be apparent from the description, figures, and claims that follow.

### BRIEF DESCRIPTION OF THE DRAWINGS

FIG. 1 depicts two representative SPIO agents (designated C5-S101 and C5-S104) that are useful for imaging conditions associated with inflammatory conditions.

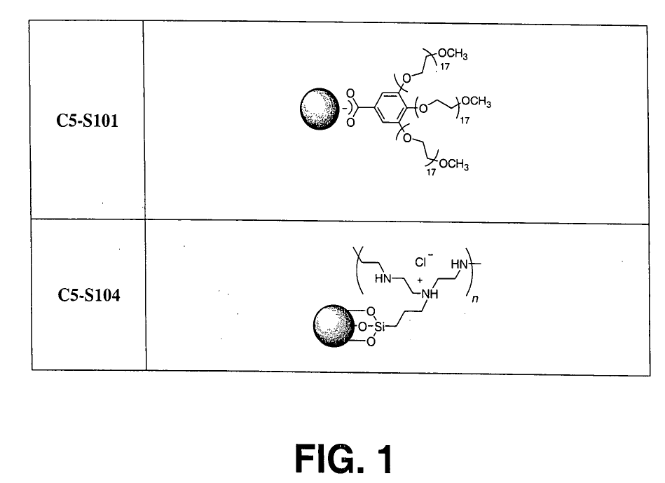

FIG. 2 depicts a one-pot synthesis method for generating the iron oxide cores for SPIO agents.

FIGS. 3A, 3B, and 3C show TEM micrographs of SPIO cores coated with lauric acid. The lower-left insert shows the spinel pattern for the SPIO agent.

FIGS. 4A, 4B, and 4C show TEM micrographs of SPIO cores of various sizes: 5 nm (FIG. 4A), 7 nm (FIG. 4B), and 9 nm (FIG. 4C).

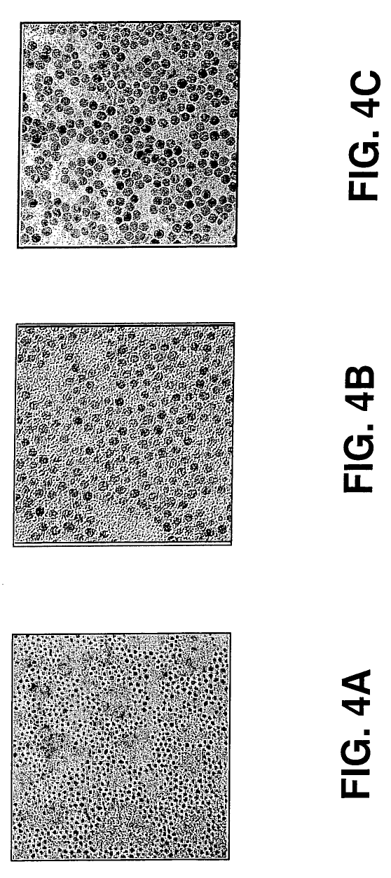

FIG. 5 depicts the macrophage cell uptake versus nanoparticle zeta potential for four SPIO agents.

FIG. 6 depicts SPIO agent uptake in the kidney of the ischemia reperfusion rat model before (FIG. 6A) and after (FIG. 6B) administration of the SPIO agent of the invention. FIG. 6A shows the same area before administration. As shown in FIG. 6B the outer medulla of the kidney in the upper portion of FIG. 6B is shows a decrease in signal intensity relative to the pre-administration kidney. Furthermore, the kidney in the lower portion of FIG. 6B, acting as an internal control, remains unchanged after administration of the SPIO agent.

FIGS. 7A and 7B depict the magnetic resonance signal intensity in the rat brain MS model. FIG. 7A shows the T2 image of brain of a normal DA rat. FIG. 7B shows a 24 hour post injection image of an EAE DA rat injected with C5-S104 at 1 mg Fe/kg bw. FIG. 7B shows a reduction in signal intensity in the cerebellum, the medulla, and the brain stem regions (circles) indicating accumulation of SPIO.

### DETAILED DESCRIPTION OF THE INVENTION

The present invention is based, in part, upon the discovery that SPIO agents may be optimized for uptake by inflammatory response cells. In particular, cationic, soluble, non-agglomerated SPIO agents are shown to be useful for imaging conditions associated with inflammatory response cell infiltration and accumulation. Methods of synthesizing SPIO agents optimized for inflammatory response cell uptake, methods for imaging inflamed tissue, and methods for managing inflammatory conditions are also disclosed herein.

Therefore, in one aspect, the present invention also provides methods for efficiently introducing SPIO agents into inflammatory response cells. In some embodiments, the SPIO agent is introduced into the inflammatory response cells ex vivo. In other embodiments the SPIO agent is directly introduced into the subject's body (in vivo) where endogenous inflammatory response cells that are located at or near the site of inflammation or are located away from the site of inflammation take up the SPIO agent and home to the site of inflamed tissue.

In another aspect, the present invention also provides for methods of imaging conditions associated with inflammatory response cell infiltration and accumulation. In yet another aspect, the present invention also provides for methods of managing diseases associated with infiltration and accumulation of inflammatory response cells using the imaging methods disclosed herein.

Preferred embodiments and exemplifications of the present invention are described in detail below.

### Definitions

To more clearly and concisely describe and point out the subject matter of the claimed invention, the following definitions are provided for specific terms, which are used in the following description, and the appended claims.

As used herein, the phrase “blood half-life” refers to the time required for the plasma concentration of an agent to decline by one-half when elimination is first-order.

As used herein, the phrase “blood residence time” refers to the amount of time that an agent remains in the blood. One measurement of blood residence time is the blood half-life of the agent. Blood residence time may also be measured by determining the time required for the relaxivity of a subject to return to base-line value following administration of a SPIO agent. Blood residence time may be modified by altering the composition of the shell, for example, by adjusting the size of the agent and/or lengthening or reducing the chain length of a polymeric component of a shell.

As used herein, the phrase “condition associated with macrophage accumulation” refers to physiological conditions wherein disease or injury causes macrophage cells to migrate to the affected tissue and accumulate at the location of disease or injury. Conditions associated with macrophage accumulation may include, for example, ischemia-reperfusion, atherosclerosis, renal failure, endometriosis, and autoimmune diseases such as multiple sclerosis and rheumatoid arthritis.

As used herein the term “core size” refers to the outer diameter (assuming a substantially spherical core) as measured by transmission electron microscopy (TEM). In some embodiments the core size is consistent in a sample, with a distribution of less than about 15%.

As used herein the term “disease management” refers to medical attention to disease conditions associated with macrophage accumulation that may be facilitated using information derived from magnetic resonance imaging. Disease management includes decisions made by medical professionals regarding the course of treatment for a subject afflicted with an inflammatory disease, including without limitation, the success or failure of a treatment, the status of the inflamed tissue, and/or whether chemical or surgical intervention is indicated. Where surgical or other non-systemic intervention is indicated, disease management also includes spatial localization of the inflamed tissue.

As used herein, the phrase “ex vivo” with regard to the introduction of SPIO agents to a body refers to processes for obtaining and manipulating cells obtained from a subject outside the subject's body. In some embodiments, ex vivo processing includes removing inflammatory response cells from a subject's body, introducing SPIO agents into the inflammatory response cells, and reintroducing the inflammatory response cells containing the SPIO agents into the subject's body. In some embodiments, the inflammatory response cells that are removed from the subject may be enriched (e.g., by sorting, magnetic bead separation, or fractionation) for a particular inflammatory response cell or particular inflammatory response cells.

As used herein the term “granularity” refers to a cellular condition characterized by the appearance of granules, which is indicative of SPIO uptake. Granularity may be measured by flow cytometry or by microscopic analysis of cells following staining with a dye, such as Prussian blue.

As used herein, the terms “hydrodynamic diameter,” “hydrodynamic size,” and the abbreviation “D_{H}” refer to the diameter of spherical particle that would have a diffusion coefficient equal to that of the nanoparticle as measured by dynamic light scattering (DLS). D_{H} values may vary depending on the medium in which the agent being measured is dispersed. Thus, unless otherwise indicated, the D_{H} values described herein were measured using DLS where the agent is dispersed in water.

As used herein, with regard to the introduction SPIO agents to a body, the phrase “in vivo” refers to methods for directly administering the disclosed SPIO agents to the subject's body under conditions where endogenous inflammatory response cells take up the SPIO agents within the subject's body. The agents of the present invention or their pharmaceutically acceptable salts can be administered to the subject in a variety of forms adapted to the chosen route of administration.

As used herein, the term “inflamed tissue” includes tissues that have elevated inflammatory response cells infiltrates. Inflamed tissue may be characterized by one or more of the following: (1) dilation of capillaries to increase blood flow to the affected area; (2) changes in the microvasculature structure, leading to the escape of inflammatory response cells from circulation; and/or (3) inflammatory response cells emigration from the capillaries and accumulation at the site of inflammation.

As used herein, the term “inflammatory response cell” refers to those cells that are stimulated by an immune response, whether the immune response results from injury, foreign antigen(s), and/or self antigen(s). Thus, inflammatory response cells include monocytes, macrophage, dendritic cells (DCs), polynuclear monocytes (PNMs), eosinophils, neutrophils, and T cells.

As used herein, the term “oxidant” generally refers to compounds that give up oxygen easily, removes hydrogen from another compound, or attracts electrons. Specific oxidants that may be used in the synthesis methods of the invention may include mild oxidants, such as trimethylamine-N-oxide. As one of ordinary skill in the art would appreciate, other oxidants similar to trimethylamine-N-oxide may also be used in the synthetic methods of the invention such as 2,6-lutidinium-N-oxide.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Any conventional media or agent that is compatible with the active ingredient can be combined with the contrast agents of the invention. Supplementary active ingredients can also be incorporated into the compositions.

As used herein the term “polydispersity” generally refers to variability of component size within a given sample. The polydispersity of the nanoparticle cores may be shown by transmission electron microscopy (TEM) of the imaging of the agent. The dispersity values for the nanoparticle cores reported herein are the standard deviation from a statistical analysis of the iron oxide cores by TEM image analysis. The polydispersity of the disclosed SPIO agents (i.e., core plus shell) may be measured using dynamic light scattering to measure the hydrodynamic diameter (D_{H}).

As used herein the term “R1 relaxivity” of an agent refers to the increase in the relaxation rate the longitudinal relaxation rate is the reciprocal of T1, the relaxation time. In specific embodiments the R1 relaxivity for SPIO agents, at room temperature and under a 1.5 Tesla field, ranges from about from about 2 mM⁻¹s⁻¹ to about 20 mM⁻¹s⁻¹.

As used herein the term “R2 relaxivity” of an agent refers to the longitudinal relaxation rate equal to reciprocal of T2 relaxation time. In specific embodiments the R2 relaxivity for SPIO agents, at room temperature and under a 1.5 Tesla field, ranges from about 10 mM⁻¹s⁻¹ to about 100 mM⁻¹s⁻¹.

As used herein, the term “R2/R1” refers to the ratio of relaxivities. R2/R1 may be used to quantify the type of contrast produced by a SPIO agent. R2/R1 may determine whether a material is useful as either a positive or negative agent or as a negative contrast agent only. Materials with R2/R1 ratios between 1 and 10 can function as either a positive or negative contrast agent depending on their concentration and the method used to acquire the magnetic resonance signal. In contrast, materials with R2/R1 greater than about 10 are primarily useful as negative contrast agents.

As used herein, the term “saturation magnetization” and the abbreviation “Mₛₐₜ” refer to the maximum possible magnetization of a material as determined by application of sufficient magnetic field strength to saturate the material. In embodiments wherein the core is substantially composed of γFe₂O₃ the Mₛₐₜ (determined using a vibrating sample magnetometer (VSM)) is around 104 emu/g Fe. In embodiments wherein the core is substantially composed of Fe₃O₄ the Mₛₐₜ (determined by VSM) is around 127 emu/g Fe.

As used herein the term “soluble” generally refers to the ability of a substance to form a solution with another substance. Solubility is a highly desirable characteristic for SPIO agents because increased solubility lowers the polydispersity of the SPIO agents. Furthermore, soluble agents are typically less toxic and more readily integrated into biocompatible solutions than non-soluble agents. Solubility may be measured by techniques known in the art, for example, by adding the maximum amount of the agent to a solvent such as water under at a specific temperature and measuring the concentration. The high solubility of the SPIO agents of the invention allows the production of highly concentrated solutions, keeping the volume burden of the circulation within acceptable limits and compensating for dilution by bodily fluid.

As used herein, the term “SPIO agent size” refers to the D_{H} of entire SPIO agent comprising both core and the coating as measured by D_{H}. Unless otherwise indicated, all D_{H} values disclosed herein are measured using the agent dispersed in water.

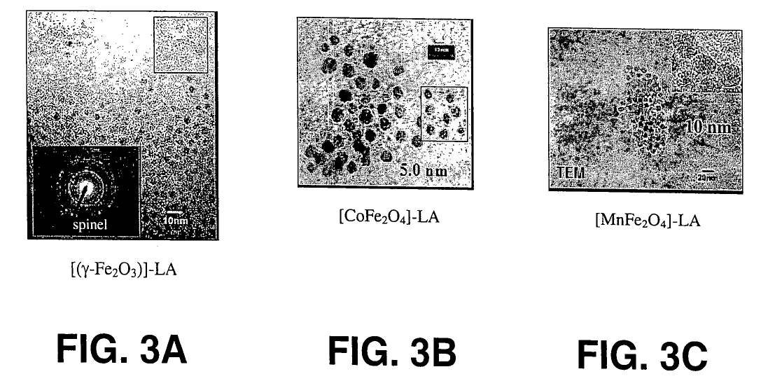

As used herein, the term “SPIO agent” refers to superparamagnetic iron oxide crystalline structures that have the general formula [Fe₂⁺O₃]ₓ[Fe₂⁺O₃(M²⁺O)]₁₋ₓ where 1≧x≧0. M²⁺ may be a divalent metal ion such as iron, manganese, nickel, cobalt, magnesium, copper, or a combination thereof. When the metal ion (M²⁺) is ferrous ion (Fe²⁺) and x=0, the SPIO agent is magnetite (Fe₃O₄), and when x=1, the SPIO agent is maghemite (□-Fe₂O₃). In general, superparamagnetism occurs when crystal-containing regions of unpaired spins are sufficiently large that they can be regarded as thermodynamically independent, single domain particles called magnetic domains. These magnetic domains display a net magnetic dipole that is larger than the sum of its individual unpaired electrons. In the absence of an applied magnetic field, all the magnetic domains are randomly oriented with no net magnetization. Application of an external magnetic field causes the dipole moments of all magnetic domains to reorient resulting in a net magnetic moment. Preferred SPIO agents demonstrate a spinel crystalline structure as shown by transmission electron microscope (TEM) analysis. A representative TEM image depicting a spinel crystalline structure is shown in the inset of FIG. 3A.

As used herein, the term “surfactant” refers to soluble compounds that reduce the surface tension between two liquids or a liquid and a solid. Specific surfactants that may be used in the synthesis methods of the invention may include lauric acid or oleic acid. As one of ordinary skill in the art would appreciate, other surfactants similar to lauric acid or oleic acid may also be used in the synthetic methods of the invention.

As used herein, the term “treating” is intended to embrace both chemical and physical medical interventions. The term “treatment” thus refers to the administration of agent (e.g., an anti-inflammatory agent and/or an antiproliferative agent) and/or the application of a treatment (e.g., radiation therapy or surgery) intended to cure or ameliorate the symptoms of an inflammatory condition.

As used herein the terms “zeta potential,” “surface potential,” and “surface charge” and the abbreviation “ζ” refers to a measurement of the electrostatic potential near the surface of the particle. As the zeta potential is affected by the solvent and ionic strength of the solvent, all zeta potential values reported herein are measured using water as the solvent unless otherwise indicated. Thus, the cationic SPIO agents of the invention display a zeta potential of about between about 0 and about +60 mV.

### Specific Embodiments

In one aspect, the present invention depends upon the discovery that SPIO agents may be optimized for efficient uptake by inflammatory response cells (e.g., monocytes circulating in the blood, macrophage cells in tissue, dendritic cells (DCs), polynuclear monocytes (PNMs), eosinophils, and T cells) to facilitate imaging of inflamed tissue and manage conditions associated with infiltration and accumulation of inflammatory response cells. Representative conditions associated with the infiltration and accumulation of inflammatory response cells may include autoimmune disease, vascular disease, and neurological diseases.

Thus, in a first series of embodiments, the present invention provides methods of imaging an inflammatory condition in a mammal comprising introducing a SPIO agent including a superparamagnetic core and a cationic coating into inflammatory cells in vivo or ex vivo, permitting the inflammatory cells to migrate to inflamed tissue, and imaging the inflamed tissue using magnetic resonance.

In some embodiments the SPIO agent is a cationic, soluble, polydisperse SPIO agent. The SPIO agents of the invention may or may not comprise a superparamagnetic inner core divalent metal ion. When the core is composed entirely of Fe₂O₃, there is not divalent metal ion present. The divalent metal iron may be iron, manganese, nickel, cobalt, magnesium, copper or a combination thereof. The size of the core may be about 2 nm to about 200 nm, about 2 nm to about 100 nm, or about 5 nm to about 9 nm. In some other embodiments the size of the core is about 9 nm, about 7 nm, or about 5 nm. In some embodiments, the combined hydrodynamic size (D_{H}) of the inner core and coating is about 3 nm to about 25 nm. In other embodiments the combined D_{H} of the inner core and coating is about 17 nm.

The SPIO agents of the invention include a surface coating substantially comprised of PEG, PEI, or combinations thereof. When the surface coating includes PEG, it may comprise PEG-silane, PEG-dendron, silane-PEG, or combinations thereof. In embodiments employing dendritic structures, the branching pattern may be limited to generation-0 (i.e., a single branch). In alternate embodiments, the dendritic structures may include multiple branches,

In some embodiments, the SPIO agent of the invention is positively charged with zeta potential greater than 0 and less than about +60 mV, about +20 mV to about +40 mV. In some other embodiments, the zeta potential of the agent is about +40 mV.

The SPIO agents of the invention are preferably non-agglomerated with a polydispersity of less than about 15% as determined by TEM. In some embodiments, non-agglomerated SPIO agents are capable of passing through a membrane with a 100 kDa cut-off value.

In some embodiments, the R1 relaxivity of the agent is greater than about 4 mM⁻¹s⁻¹ and the R2 relaxivity of the agent is greater than about 20 mM⁻¹s⁻¹. In other embodiments, the R2/R1 ratio of the agent is greater than about 2.

The SPIO agents of the invention may be dispersed in physiologically acceptable carrier to minimize potential toxicity. Thus, the SPIO agents of the present invention may be dispersed in a biocompatible solution with a pH of about 6 to about 8. In some embodiments, the agent is dispersed in a biocompatible solution with a pH of about 7 to about 7.4. In other embodiments, the agent is dispersed in a biocompatible solution with a pH of about 7.4. In addition, the SPIO agents of the invention show not only a high stability in vitro but also high stability in vivo, so that a release or an exchange of the ions, which are inherently toxic and not covalently bonded in the complexes, will not be harmful within the time that it takes for the contrast media to be completely excreted from the body of the subject.

The SPIO agents of the invention may be combined with additives that are commonly used in the pharmaceutical industry to suspend or dissolve the compounds in an aqueous medium, and then the suspension or solution can be sterilized by techniques known in the art. The agents of the present invention or their pharmaceutically acceptable salts can be administered to the subject in a variety of forms adapted to the chosen route of administration. Thus, the SPIO agents of the invention may be topically (i.e., by the administration to the tissue or mucus membranes), intravenously, intramuscularly, intradermally, and/or subcutaneously. Forms suitable for injection include sterile aqueous solutions or dispersions and sterile powders for the preparation of sterile injectable solutions, dispersions, liposomal, or emulsion formulations. In all cases, the form must be sterile and should be fluid to enable administration by a syringe. Forms suitable for inhalation use include SPIO agents dispersed in a sterile aerosol. Forms suitable for topical administration include creams, lotions, ointments, and the like.

In some embodiments, the SPIO agents of the invention are concentrated to conveniently deliver a preferred amount of the SPIO agents to a subject and packaged in container in the desired form. Thus, in some embodiments the SPIO agent is dispensed in a container dispersed in physiologically acceptable solution, that conveniently facilitates administering the SPIO agent in concentrations of about 0.1 mg of Fe content of the agent per kg body weight of the subject (i.e., 0.1 mg Fe/kg bw) to about 50 mg Fe/kg bw. In other embodiments, the SPIO agent is packaged in a manner that conveniently facilitates administration of the SPIO agent in concentrations of about 0.5 mg Fe/kg bw to about 2.5 mg Fe/kg bw.

In one series of embodiments, the disclosed SPIO agents may be administered directly to the subject in a variety of ways including topically, intravascularly, intramuscularly, or interstitially. In some embodiments, about 0.1 mg Fe/kg to about 50 mg Fe/kg of SPIO agent is administered to the subject. In other embodiments, about 0.5 mg Fe/kg to about 2.5 mg Fe/kg of agent is administered to the subject. Similarly, inflammatory response cells containing of the disclosed SPIO agents may be administered to the subject in a variety of ways including intravascularly, intramuscularly, or interstitially.

In some embodiments, the target tissue is imaged less than or approximately 3 hours after administering the SPIO agents or inflammatory response cells containing the SPIO agents. In alternative embodiments, the target tissue is imaged less than or approximately 24 hours after administering to the subject the SPIO agents or inflammatory response cells containing SPIO agents. In other alternative embodiments, target tissue is imaged less than or approximately 5 days after administering to the subject the SPIO agents or inflammatory response cells containing SPIO agents

In another series of embodiments, the present invention provides for methods of imaging conditions associated with inflammatory response cells infiltration and accumulation using the SPIO agents of the invention. The SPIO agents of the present invention may be introduced into inflammatory response cells ex vivo and subsequently introduced into the subject. Thus, the inflammatory response cells may be withdrawn from the subject, the SPIO agent introduced into the inflammatory response cells, and the inflammatory response cells containing the SPIO agent are administered to subject prior to imaging. The step of introducing the SPIO agents into the inflammatory response cells may optionally include the step of separating the inflammatory response cells using magnetic beads, density agents and/or centrifugation. In certain embodiments, the inflammatory response cells comprise monocytes circulating in the blood, macrophage cells in tissue, dendritic cells (DCs), polynuclear monocytes (PNMs), eosinophils, neutrophils, and T cells.

The methods of managing conditions associated with inflammatory response cell infiltration and accumulation may include imaging the target tissue before, after, or both before and after treating the subject to reduce inflammation. Thus, the disclosed methods of managing conditions associated with inflammatory response cell infiltration and accumulation may include (a) imaging the target tissue to obtain base-line or diagnostic information about an inflammatory condition, (b) treating the subject, and (c) imaging the subject a one or more times to obtain further information about the inflammatory condition. A medical professional may opt not to image the subject both before and after treatment, relying on other techniques to initially characterize the inflamed tissue or subsequently assess the inflamed tissue. Thus, in an alternative embodiment, the methods of managing conditions associated with inflammatory response cell infiltration and accumulation includes treating an inflammatory condition that was identified by a technique other than magnetic resonance and imaging the target issue subsequent to treatment. Likewise, in another alternative embodiment, the disclosed methods of managing conditions associated with inflammatory response cell infiltration and accumulation may include imaging a subject or target tissue to obtain information about an inflammatory condition followed by treating the inflammatory condition without subsequently re-imaging the target tissue.

When the disease management is directed to determining the efficacy of a treatment, the methods comprise imaging the tissue of interest before administration of a treatment to obtain a pre-treatment assessment, followed by administration of the treatment and imaging the tissue of interest one or more times subsequent to the treatment to obtain a post-treatment assessment of the tissue of interest. The pre-treatment assessment and the post-treatment assessment(s) may be compared to determine whether the reduced inflammation or otherwise ameliorated the symptoms of the condition associated with inflammatory response cells infiltration and accumulation. The methods of determining the efficacy of a treatment may further comprise deciding whether to cease a particular treatment, as well as decisions to increase the frequency, intensity, and/or dose of a treatment based on the comparison of the pre- and post-treatment assessments.

When the disease management includes treatments that are localized to the inflamed tissue rather than a holistic or systemic administration of treatment (e.g., surgical or radiological intervention), the disease management methods may include determining the spatial localization of the inflamed tissue to define the specific area to be treated (e.g., excised or irradiated).

### EXAMPLES

Practice of the invention will be still more fully understood from the following examples, which are presented herein for illustration only and should not be construed as limiting the invention in any way.

Nanocore Synthesis

General Synthesis of Nanocrystal Core

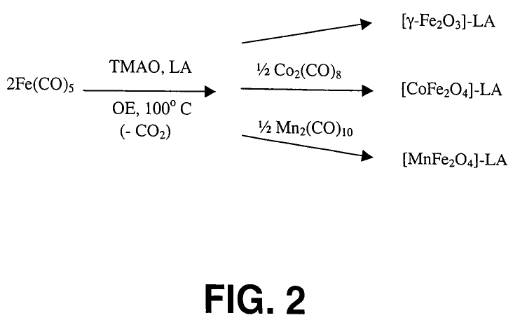

Provided herein are synthetic methods based on organometallic chemistry that generate soluble, crystalline, and monodisperse magnetic nanoparticles in a one-pot reaction. High-temperature oxidative decomposition of iron pentacarbonyl, used exclusively or in combination with various first-row transition metal carbonyls, are used to generate unagglomerated, superparamagnetic (spinel ferrite) crystalline nanoparticles in the presence of surfactant and a mild oxidant in one-pot as shown in FIG. 2.

Using the disclosed methods, organically soluble magnetic nanoparticle cores containing a single metal (e.g., γ-Fe₂O₃/Fe₃O₄ spinel) or multiple metals (e.g., MnFe₂O₄ spinel, Mn-ferrite) may be produced. The non-water-soluble cores are rendered water soluble by the application of the hydrophilic shells disclosed herein, a desirable characteristic for any agent to be introduced into a living cell or subject.

In the synthesis of the nanoparticle cores, lauric acid and oleic acid may be used a surfactants, and trimethylamine-N-oxide may be used as the oxidant. The solvent of may be dioctyl ether or hexadecane. However, as the skilled artisan would recognize, other surfactants, oxidants, and/or solvents may be used in place of these specific agents. Heating the solution to about 100° C. during the first stage of this two-stage method facilitates solublization the surfactant and trimethylamine-N-oxide in dioctyl ether. Elevated second-stage temperature (˜260-280° C.) results in synthesis of tractable and crystalline nanomaterials. Higher temperatures at the second stage and/or longer reaction times during the second stage of this two-stage heating process tend to increase particle sizes beyond the target core size. Thus, in some methods of synthesis, the second stage temperature may be capped at about 290° C. and the first stage second stage total reaction time may be limited to between about 180 and about 240 minutes, as excessive heating temperatures and times may increase particle sizes beyond the target size.

FIGS. 3A, 3B, and 3C shows representative TEM micrographs of Mn— and Co-nanoferrites, obtained by the general method set out in FIG. 2 wherein Fe(CO)₅ was decomposed to yield γ-Fe₂O₃/Fe₃O₄ in presence of other metal carbonyls. Resultant specimens analyzed by selected area electron diffraction (SAED) proved to be cubic phase material (spinel structured) as evidenced by indexed diffraction patterns.

Synthesis of γ-Fe₂O₃/Fe₃O₄ nanoparticles. In the absence of a second transition-metal carbonyl, the ‘all-Fe’ system results in the formation of SPIO nanocrystals. Charging a Schlenk flask under N₂ with Fe(CO)₅, containing lauric acid (or oleic acid), trimethylamine-N-oxide, and dioctyl ether at ˜100° C., initiates a vigorous and mildly exothermic reaction. (CO₂ and trimethylamine are likely liberated upon injection of iron pentacarbonyl into the homogeneous solution.) The mixture is allowed to stir for 75 minutes at 100° C. to completely decompose remaining Fe(CO)₅, then heated to (at least) ˜260° C. for ˜1 hour at which point the solution looked deep-brown/black. The mixture is allowed to cool, and almost twice the volume of acetonitrile added. After centrifugation and decantation of the supernatant, crude SPIO containing excess surfactant in dioctyl ether, may be diluted (in roughly twice its volume) with tetrahydrofuran for ligand exchange (shell) chemistry. Residual the dioctyl ether can be removed by high-vacuum distillation without changing the size of the particles.

To obtain precipitates for solids analysis (using, for example, VSM or ICP-AES) isopropyl alcohol (instead of acetonitrile) gave deposition of the particles. After centrifugation and decantation of the alcoholic supernatant, crude SPIO was observed as a pellet (brown sludge) at the bottom of the centrifuge tube. Repeated excessive washings with isopropyl alcohol, ethanol, methanol, or acetone removed all surfactant, caused irreversible agglomeration, and spoiled further shell (derivatization) chemistry. The resulting powder was used for elemental analysis and in magnetic studies (vibrating sample magnetometry).

The synthesis described above involves a two-stage heating process. The Fe(CO)₅ is added to a warm reaction mixture (˜80° C.) to encourage partial CO loss, thereby creating CO-deficient (and perhaps polar) Fe species, and stirred for 75 minutes at 100° C. to decompose any remaining iron pentacarbonyl. Immediately following the low temperature stage, an increase in reaction temperature to ˜280° C. is used to generate highly-crystalline SPIO.

Synthesis of 5 nm SPIO nanoparticle. A 25 mL, 3-neck Schlenk flask was fitted with a condenser, stacked on top of a 130 mm Vigreux column, and a thermocouple. The condenser was fitted with a nitrogen inlet and nitrogen flowed through the system. The Schlenk flask and Vigreux column were insulated with glass wool. Trimethylamine-N-oxide (Aldrich, 0.570 g, 7.6 mmol) and oleic acid (Aldrich: 99+%, 0.565 g, 2.0 mmol) were dispersed in 10 mL of dioctylether (Aldrich: 99%). The dispersion was heated to 80° C. at a rate of about 20° C./minutes. Once the mixture had reached ˜80° C., 265 μL of Fe(CO)₅ (Aldrich: 99.999%, 2.0 mmol) was rapidly injected into the stirring solution through the Schlenk joint. The solution turned black instantaneously, with a violent production of a white “cloud.” The solution rapidly heated to ˜120-140° C. Within 6-8 minutes the reaction pot cooled to 100° C. at which it was kept and stirred for 75 minutes. After stirring at ˜100° C. for 75 minutes, the temperature was increased to ˜280° C. at a rate of about 20° C./min. After the solution stirred for 75 minutes, the heating mantel and glass wool were removed to allow the reaction to return to room temperature. Once at room temperature, an aliquot was removed and dissolved in toluene for size measurement using dynamic light scattering (DLS), image analysis using transmission electronic microscopy (TEM), and elemental analysis using energy dispersive x-ray analysis (EDX).

Nanoparticle Core Characterization.

To prepare a sample for vibrating sample magnetometer analysis and elemental analysis approximately 5-10 mL of crude reaction solution was added to 20 mL of isopropanol, and the solution was centrifuged for 10 minutes at 3000 rpm. The supernatant was decanted, an additional 20 mL of isopropanol was added, and again the precipitate was collected by centrifugation. The precipitated iron oxide nanoparticles were allowed to air-dry overnight, yielding a black magnetic powder.

Saturation Magnetization. The saturation magnetization (Mₛₐₜ) of the precipitated SPIO nanoparticles was measured using a vibrating sample magnetometer (VSM). Elemental analysis was performed on the magnetic powder to determine the concentration of Fe, and the Mₛₐₜ was calculated in units of emu/g Fe for each sample. The Mₛₐₜ for bulk γ-Fe₂O₃ and Fe₃O₄ is known to be ˜104 emu/g Fe and ˜127 emu/g Fe, respectively. Although some reactions yielded SPIO agents with Mₛₐₜ values lower than 100 emu/g Fe, Mₛₐₜ values for the disclosed SPIO agents typically ranges from about 100 emu/g Fe to about 120 emu/g Fe.

Nanoparticle Shell Synthesis

To impart water solubility to the oleic acid-ligated SPIO nanoparticles obtained from the nanoparticle synthesis, the oleic acid is displaced with small molecule organic ligands containing water-soluble functionality. Small molecule organic ligands useful for this purpose include: water soluble silane, carboxylic acids, sulfonic acids, phoshonic acids, and alcohols. These small molecule organic ligands provide water solubility and also prevent nanoparticle aggregation through either steric and/or electrostatic repulsions. The water-soluble shells these ligands form around the nanoparticles are held in place through either ionic interactions or covalent bonds, depending on the ligand structure. The ionic binding results from electrostatic interaction of the positive surface charge of the iron oxide nanoparticles with a carboxylate functionality and employ the same binding mode exhibited by the oleic acid used in the core synthesis.

This binding mode has been used for the development of the core/shell structure C5-S101. The covalently bound shells consist of silane based ligands which are linked to the nanoparticle surface through the base catalyzed condensation of surface hydroxyls with trialkoxy silanes and are significantly more stable than the ionic shells.

Synthesis of S101. The preparation of S101 is generally depicted in Scheme 1. The functionalization of methyl 3,4,5-trihydroxybenzoate with the mesolate of polyethylene glycol monomethyl ether (M_{w}=750) is carried out by heating in acetone using potassium carbonate as the proton scavenger. The reaction time is decreased from 4 to 1 day through the use of 50% aqueous tripropylmethyl ammonium chloride as a phase transfer catalyst. Purification of the resulting methyl tris-(3,4,5-PEG-750 monomethyl ether)benzoate is carried out via column chromatography. Subsequent saponification of the methyl ester proceeds smoothly with potassium hydroxide in 4:1 MeOH/H₂O.

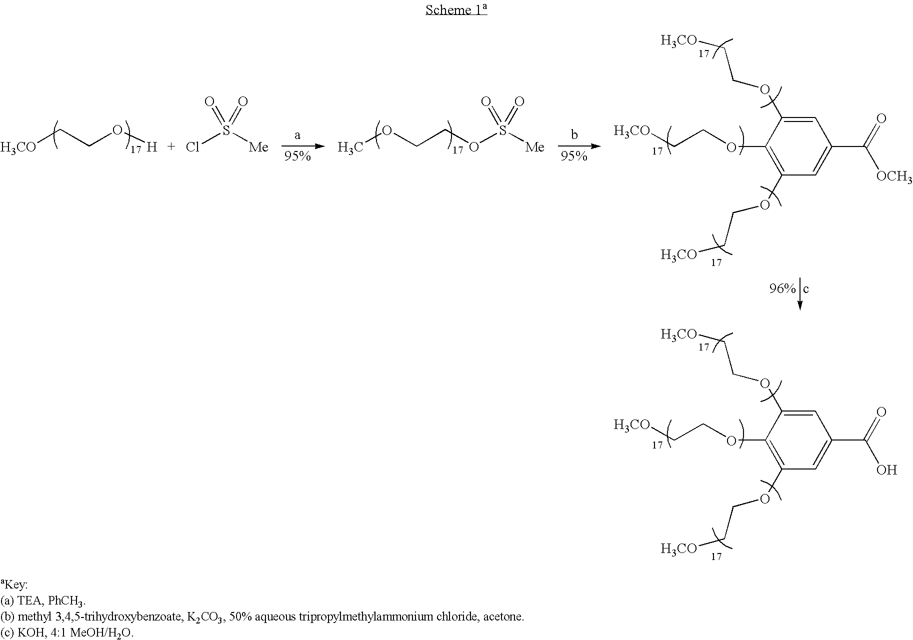

To a solution containing PEG-750 monomethyl ether (228.7 g, 305 mmol) dissolved in toluene (305 mL) was added TEA (32.33 g, 320 mmol) at 0° C. was added methane sulfonyl chloride (36.66 g, 320 mmol) was added slowly over a period of 5 min. The reaction was stirred at 0° C. for 1 h, filtered and toluene was removed in vacuo. The resulting precipitate was filtered and the filtrate removed in vacuo to leave 240.4 g (95%) of the desired product at a waxy, white solid. ¹H NMR (CD₂Cl₂) γ4.35 (m, 2H), 3.8-3.4 (m, 78H), 3.32 (s, 3H), 3.05 (s, 3H). ¹³C NMR (CD₂Cl₂) 71.8, 70.5, 70.3, 69.6, 68.9, 58.5, 37.4.

To a solution containing PEG-750 monomethyl ether methane sulfonate (3) (69.1 g, 82 mmol) acetone (110 mL) was added methyl 3,4,5-trihydroxybenzoate (5.0 g, 27 mmol), anhydrous potassium carbonate (11 g, 79.6 mmol), and 50% aqueous tripropylmethyl ammonium chloride (6.47 mL, 16.7 mmol). The resulting solution was heated to reflux for 36 h. The reaction was cooled to room temperature, filtered, and the filtrate was removed in vacuo. The crude product was purified by column chromatography (100% CH₂Cl₂ to 10% MeOH/90% CH₂Cl₂) to provide 62.2 g (95%) of the desired product as a golden colored waxy solid. ¹H NMR (CD₂Cl₂) γ7.3 (s, 2H), 4.20 (m, 6H), 3.9-3.4 (m, 229H), 3.36 (s, 9H). ¹³C NMR (CD₂Cl₂) 166.3, 152.3, 142.4, 125.0, 108.7, 72.4, 71.9, 70.7, 70.5, 70.4, 69.6, 68.8, 58.6, 51.9. MS (MALDI-TOF) m/z calcd for (M+Na)⁺ (Cl₁₁₃H₂₁₈O₅₆+Na) 2494.7, found 2495.7.

To a solution containing methyl tris-(3,4,5-PEG-750 monomethyl ether)benzoate (36.0 g; 14.8 mmol) dissolved in 4:1 methanol/H₂O (115 mL) was added potassium hydroxide (10 g, 178 mmol) and the resulting solution was stirred at room temperature for 2 hours. The reaction was quenched by acidification to pH 1 with concentrated HCl. The methanol was removed in vacuo, the resulting solution was diluted with H₂O (150 mL), and extracted with CH₂Cl₂ (3×150 mL). The organic layers were combined, washed with brine (150 mL), and dried over anhydrous MgSO₄. Filtration and removal of the solvent provided 34.4 g (96%) of the product as a pale yellow solid. ¹H NMR (CD₂Cl₂) δ 7.32 (s, 2H), 4.25 (m, 6H), 3.9-3.4 (m, 225H), 3.36 (s, 9H). ¹³C NMR (CD₂Cl₂) 167.3, 152.3, 142.5, 125.1, 109.0, 72.5, 72.4, 71.9, 70.7, 70.5, 70.34, 69.6, 68.9, 58.6. MS (MALDI-TOF) m/z calculated for (C₁₁₃H₂₁₈O₅₆+Na) 2480.7, found 2480.6.

Synthesis of S102

The synthesis of S102 may be performed by coupling of PEG-750 monomethyl ether (M_{w}=750) with isocyanatopropyltrimethoxy silane in the presence of triethyl amine as a catalyst (Scheme 2). The reaction is carried out in CH₂Cl₂ and its progress is easily followed via IR analysis of the crude reaction solution. Loss of the isocyanide stretch at 2273 cm⁻¹ indicates complete reaction. Due to the hydrolytic liability of the trimethoxy silane moiety, the triethyl amine is removed in vacuo and material is then reacted with the SPIO nanoparticles. The reaction time can be decreased through the use of higher concentrations of TEA, although, under these conditions, removal of the TEA from the product becomes more difficult.

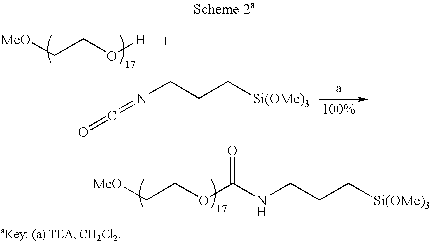

To a solution containing PEG-750 monomethyl ether (21.776 g, 28.5 mmol) dissolved in CH₂Cl₂ (100 mL) was added isocyanatopropyl trimethoxysilane (5.322 g, 25.9 mmol) followed by triethylamine (0.866 g, 8.55 mmol). The resulting solution was stirred at room temperature until IR analysis showed complete consumption of the isocyanate stretch at 2273 cm⁻¹ (˜24 hours). The solvent was then removed in vacuo to leave 25.13 g (100%) of the product as a white, waxy solid. ¹H NMR (CD₂Cl₂) δ 4.13 (t, J=8.0 Hz, 2H), 3.75-3.46 (m, 75H), 3.31 (s, 3H), 3.08 (m, 2H), 1.54 (m, 2H), 0.58 (m, 2H). ¹³C NMR (CD₂Cl₂) δ 73.0, 72.4, 71.0, 70.8, 70.7, 62.0, 59.1, 50.8, 24.1, 6.7. IR (neat on salt plate) 2870, 1717, 1652, 1456, 1249, 1107, 817 cm⁻¹.

Synthesis of S103

The preparation of S103 is carried out through functionalization of methyl 3,4,5-trihydroxybenzoate with the mesolate of polyethylene glycol monomethyl ether (M_{w}=550) followed by subsequent hydrolysis of the methyl ester and coupling 3-aminopropyl triethoxy silane (Scheme 3). The coupling of methyl 3,4,5-trihydroxybenzoate with the PEG-550 mesolate is carried out under identical conditions to those employed for the synthesis of S101. Subsequent hydrolysis of the methyl ester proceeds smoothly with potassium hydroxide and coupling of the resulting acid with 3-aminopropyltriethoxy silane is easily carried out via the acyl chloride in the presence of excess TEA. Due to the increased hydrolytic stability of the triethoxy silane as compared to the trimethoxy silane used in the synthesis of S102 purification is carried out by column chromatography prior to reaction with the SPIO nanoparticles.

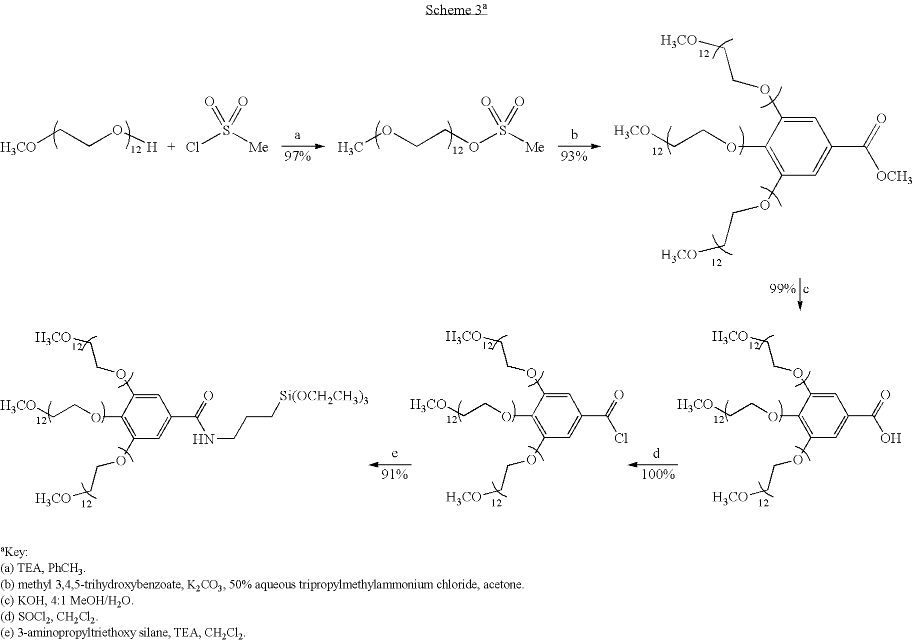

To a solution containing PEG-550 monomethyl ether (20.76 g, 38.2 mmol) dissolved in toluene (38.0 mL) was added triethyl amine (4.05 g, 40.1 mmol). The resulting solution was cooled to 0° C. and methane sulfonyl chloride (4.59 g, 40.1 mmol) was added slowly over a period of 5 min. The solution was then stirred at 0° C. for 1.5 h. The resulting precipitate was filtered and the filtrate removed in vacuo to leave 23.04 g (97%) of the product as a yellow oil. ¹H NMR (CD₂Cl₂) δ 4.36 (m, 2H), 3.8-3.4 (m, 50H), 3.35 (s, 3H), 3.07 (s, 3H) ¹³C NMR (CD₂Cl₂) δ 71.9, 70.6, 70.5, 70.4, 70.3, 69.6, 68.9, 5 8.5, 37.5.

Synthesis of methyl tris-(3,4,5-PEG-550 monomethyl ether)benzoate. To a solution containing PEG-550 monomethyl ether methane sulfonate (4.952 g, 7.46 mmol) dissolved in acetone (50.0 mL) was added methyl 3,4,5-trihydroxybenzoate (0.772 g, 4.19 mmol), anhydrous potassium carbonate (2.70 g, 19.5 mmol) and 50% aqueous tripropylmethyl ammonium chloride (1.0 mL, 2.58 mmol). The resulting solution was heated to reflux for 36 h. The reaction was cooled to room temperature, filtered, and the filtrate was removed in vacuo. The crude product was purified by column chromatography (100% CH₂Cl₂ to 10% methanol/90% CH₂Cl₂) to afford 7.13 g (93%) of the product as a waxy, pale yellow solid. ¹H NMR (CD₂Cl₂) δ 7.30 (s, 2H), 4.20 (m, 6H), 3.9-3.4 (m, 147H), 3.35 (s, 9H). ¹³C NMR (CD₂Cl₂) δ 166.3, 152.3, 142.4, 125.0, 108.7, 72.4, 71.9, 70.7, 70.5, 70.4, 69.6, 68.8, 58.6, 51.9. MS (FAB+) m/z calculated for (M+H)⁺ (C₈₀H₁₅₂O₃₈+1H) 1722, found 1723.

To a solution containing methyl tris-(3,4,5-PEG-550 monomethyl ether)benzoate (6.07 g, 3.52 mmol) dissolved in 4:1 methanol/H₂O (35 mL) was added potassium hydroxide (2.37 g, 42.3 mmol) and the resulting solution was stirred at room temperature for 2 hours. Adjusting the pH to 1 with concentrated HCl quenched the reaction. The methanol was removed in vacuo, the resulting solution was diluted with H₂O (30 mL), and extracted with CH₂Cl₂ (3×50 mL). The organic layers were combined, washed with brine (50 mL), and dried over anhydrous MgSO₄. Filtration and removal of the solvent provided 5.95 g (99%) of the product as a pale yellow solid. ¹H NMR (CD₂Cl₂) δ 7.35 (s, 2H), 4.25 (m, 6H), 3.9-3.4 (m, 141H), 3.36 (s, 9H). ¹³C NMR (CD₂Cl₂) δ 167.8, 152.2, 142.6, 124.9, 109.1, 72.4, 71.9, 70.7, 70.46, 70.31, 69.6, 68.9, 58.5. MS (FAB−) m/z calculated for (M−H)⁻ (C₇₉H₁₅₀O₃₈-1H) 1707.00, found 1707.

To a solution containing tris-(3,4,5-PEG-550 monomethyl ether)benzoic acid (2.444 g, 1.35 mmol) dissolved in CH₂Cl₂ (13.5 mL) was added thionyl chloride (0.804 g, 6.75 mmol). The resulting solution was stirred at room temperature for 2 h and the solvent was removed in vacuo to leave 2.48 g (100%) of the product a yellow oil. IR (neat on salt plate) 2878, 1749, 1582, 1453, 1266, 1106, 950, 851, 736, 701 cm⁻¹.

Synthesis of S103. To a solution containing tris-(3,4,5-PEG-550 monomethyl ether)benzoyl chloride (2.477 g, 1.35 mmol) dissolved in CH₂Cl₂ (13.5 mL) was added triethyl amine (0.711 mL, 7.02 mmol) followed by 3-aminopropyltriethoxy silane (0.285 g, 1.29 mmol). The resulting solution was stirred at room temperature for 16 h and the solvent was removed in vacuo. The crude product was purified by column chromatography (100% CH₂Cl₂ to 10% methanol/90% CH₂Cl₂) to afford 2.36 g (91%) of the desired product as a pale yellow solid. ¹H NMR (CD₂Cl₂) δ 7.05 (s, 2H), 4.20 (m, 6H), 3.9-3.3 (m, 146H), 3.33 (s, 9H), 1.68 (m, 2H), 1.20 (m, 9H), 0.66 (m, 2H). IR (neat on salt plate) 2879, 1651, 1452, 1348, 1267, 1104, 956, 851, 735, 701 cm⁻¹.

Synthesis of S104. PEI-silane is commercially available from Gelest Inc. as a 50% solution of trimethoxysilylpropyl modified (polyethylene imine) in isopropanol. The molecular weight range of the PEI is 800-1600 and the material is used as received to coat the SPIO nanoparticles.

Synthesis of S105. TMS-Glucose-silane is prepared via coupling of TMS protected D-glucono-1,5-lactone with 3-aminopropyltriethoxy silane (Scheme 4). Initial protection of D-glucono-1,5-lactone is carried out using hexamethyldisilazane and trimethylchlorosilane and is necessary to provide solubility in tetrahydrofuran (THF) for reaction with the SPIO nanoparticles. The coupling of the protected sugar and 3-aminopropyltriethoxy silane is carried out at reflux and the material is readily purified by column chromatography.

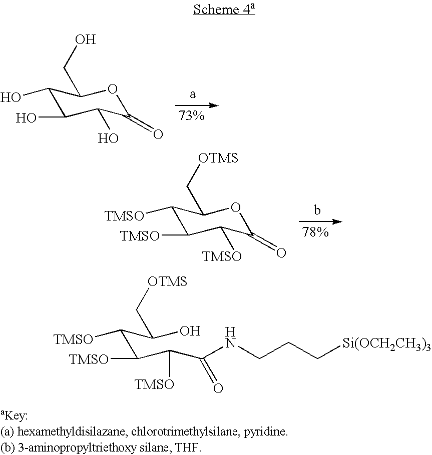

To a solution of D-glucono-1,5-lactone (17.0 g, 95.43 mmol) in dry pyridine (160 mL) were added hexamethyldisilazane (79 mL, 378.8 mmol) and chlorotrimethylsilane (25 mL, 197 mmol), and the mixture was stirred vigorously for 25 minutes at room temperature. This reaction is exothermic. Pentane (500 mL) was then added, and the white precipitate that formed was filtered off through Celite. The filtrate was evaporated, and the oil remaining was distilled under high vacuum, to give the desired product as the fraction boiling at 128-129° C. at 0.4 torr to afford 32.7 g (73%) of the desired product as a clear colorless oil. ¹H NMR (CDCl₃) δ 4.18 (m, 1H), 4.00 (d, J=7.6 Hz, 1H), 3.90 (t, J=7.1 Hz, 1H), 3.79 (m, 2H), 3.75 (m, 1H), 0.19 (s, 9H), 0.17 (s, 9H), 0.16 (s, 9H), 0.12 (s, 9H). ¹³C NMR (CDCl₃) δ 170.8, 81.3, 76.2, 73.2, 71.0, 61.5, 0.7, 0.5, 0.2, 0.4.

Synthesis of S105. To a solution containing 2,3,4,6-tetra-O-(trimethylsilyl)-D-glucono-1,5-lactone (6.640 g, 14.23 mmol) dissolved in tetrahydrofuran (15 mL) was added a solution containing 3-aminopropyltriethoxy silane (3.000 g, 13.6 mmol) dissolved in tetrahydrofuran (15 mL). The resulting solution was refluxed with vigorous stirring for 24 h. The solvent was removed in vacuo to afford a light yellow oil. The crude product was further purified by flash column chromatography (3:1 hexane/ethyl acetate) to afford 7.2 g (78%) of the desired product as clear colorless oil. ¹H NMR (CDCl₃) δ 6.47 (s, 1H), 4.31-2.98 (m, 15H), 1.52 (m, 2H), 1.09 (t, J=7.0 Hz, 9H), 0.52 (m, 2H), 0.1-−0.05 (m, 36H). ¹³C NMR (CDCl₃) δ 172.2, 75.8, 73.4, 73.3, 70.2, 63.8, 58.2, 41.4, 23.1, 18.1, 7.8, 0.24, −0.03, −0.09, −0.51.

SPIO Agent Preparation

Preparation of C5-S101. Preparation of C5-S101 is carried out via exchange of the oleic acid ligand on the surface of 5 nm SPIO nanoparticles with S101. The exchange is carried out by sonication of the oleic acid coated SPIO nanoparticles with an excess of S101 in tetrahydrofuran (THF). Purification is carried out via extraction of an aqueous solution of the nanoparticles with hexanes to remove the displaced oleic acid. Further purification aimed at the removal of the remaining excess ligand leads to nanoparticle aggregation and subsequent nanoparticle precipitation. The instability of the C5-S101 to removal of excess ligand is likely the result of an equilibrium between bound and nonbound ligand. When the ligand is present in excess the equilibrium favors the formation of C5-S101 and the SPIO remains soluble. As the excess as ligand is removed from solution (as a result of purification), the equilibrium shifts the uncoated nanoparticles aggregate and ultimately precipitate.

The preparation of C5-S101 has been assumed to be essentially quantitative given that no significant particle aggregation was observed in successful coating batches (D_{H}<40 nm) and that free iron is not removed in any way during the purification process. Successful coatings of SPIO nanoparticles have been performed on scales up to 100 mg of Fe and there are no obvious issues with further scale-up of this process aside from the above noted difficulties with reproducibly preparing this agent in a monodisperse form.

A solution containing 5 nm SPIO nanoparticles (100 mg Fe, 1.79 mmol Fe) and S101 (5.00 g, 2.02 mmol) dissolved in tetrahydrofuran (20 mL) was sonicated for 20 hours. The resulting solution was diluted with H₂O (20 mL), the tetrahydrofuran was removed in vacuo, and extracted with hexanes (3×20 mL). The aqueous layer was diluted with acetone (60 mL) and the acetone was removed in vacuo to leave an aqueous solution containing C5-S101.

Preparation of C5-S102, C5-S103, C5-S104, and C5-S105

The method of preparation of the silane coated SPIO nanoparticles is the same for all four of the silane-based coatings. The ligand exchange involves sonication of the oleic acid coated SPIO with excess trialkoxy silane for 2 hours in tetrahydrofuran (THF) followed by an additional 16 hours sonication after the addition of IPA. Covalent linkage of the silane to the SPIO surface is carried out through stirring with NH₄OH for 4 hours. Purification is performed through extraction of aqueous solutions with hexanes and EtOAc. Removal of the excess silane material may be accomplished by repeated washing through 100 KDa centrifuge filters and subsequent adjustment of the solution pH to ˜7.4-7.7 with HCl. Unlike the C5-S101, removal of excess silane does not induce nanoparticle aggregation. This is evidenced by the absence of an increase in D_{H} as the number of washes increases.

Significant multilayer formation is not likely given the relatively small increase in D_{H} upon coating the nanoparticles. For example, uncoated 5 nm SPIO nanoparticles exhibit D_{H}≈8-9 nm in tetrahydrofuran (THF) and the D_{H} for the particles coated with C5-S102 or C5-S104 are on the order of 12-14 nm in THF. This indicates that a significant quantity of free silane based ligand is present in the coated SPIO nanoparticle solutions.

To decrease the quantity of unbound silane that need to be removed during purification, the initial Si/Fe ratio was optimized. A starting Si/Fe molar ratio of about 5.5 is preferred, with higher ratios leading to larger quantities of free silane in the product and lower ratios leading to the formation of aggregated particles during the coating process.

Silane-based ligand exchange reactions using S102 and S104 were carried out in the absence of IPA with only 2 hours sonication followed by stirring at room temperature with NH₄OH. Both processes yield water-soluble nanoparticles with similar D_{H} and surface charges. However, cell viability studies using RAW 264.7 mouse macrophage cells and C5-S104 prepared without added IPA have shown a marked decrease in cell viability when compared to C5-S104 prepared with IPA. Since water-soluble nanoparticles with similar properties can be prepared in the absence of IPA, it is assumed that the IPA is facilitating the purification process. Although the toxin(s) which are removed when IPA unidentified, analysis of the centrifuge filtrate for material prepared with and without IPA show the presence of free Fe as evidenced by reaction with potassium ferrocyanate and elemental analysis.

Preparation of C5-S102. To a vial containing 3.25 mg Fe/mL 5 nm SPIO in tetrahydrofuran (4.0 mL 13 mg Fe, 0.232 mmol) was added a solution containing PEG-750 carbamate trimethoxysilane (2.337 g, 2.45 mmol) in tetrahydrofuran (10.0 mL). The resulting solution was sonicated for 2 h. Isopropanol (4.0 mL) was added and the solution sonicated for 16 hours. Concentrated NH₄OH (1.0 mL, 14.8 mmol) was then added and the solution was stirred at room temperature for 4 h. The solution was then diluted with H₂O (10.0 mL) and extracted with hexanes (3×10 mL) and etoleic acid (3×10 mL). Any remaining organics were removed in vacuo. The resulting homogeneous aqueous solution was passed through a 200 nm followed by a 100 nm syringe filter. The solution was then diluted with H₂O (10 mL total volume) and purified using a 100 kDa MW cutoff filter (2680×g until ˜3 mL of solution remained). The centrifuge filtration process was carried out a total of 6 times. The final pH of the solution was adjusted to 7.4-7.7 using concentrated HCl as necessary.

Preparation of C5-S103. To a vial containing 5.92 mg Fe/mL 5 nm SPIO in tetrahydrofuran (1.89 mL, 11.2 mg Fe, 0.200 mmol Fe) was added a solution containing G0 PEG-550 linked silane (2.22 g, 1.10 mmol) dissolved in tetrahydrofuran (10.07 mL). The resulting solution was sonicated for 2 hours. Isopropanol (3.42 mL) was then added and the solution was sonicated for an additional 16 hours. Concentrated NH₄OH (0.854 mL, 12.6 mmol) was then added and the solution was stirred at room temperature for 4 hours. The solution was then diluted with H₂O (5 mL) and extracted with hexanes (3×5 mL) and etoleic acidic (3×5 mL). Any remaining organics were removed in vacuo. The resulting homogeneous aqueous solution was passed through a 200 nm followed by a 100 nm syringe filter. The solution was then diluted with H₂O (10 mL total volume) and purified using a 100 kDa MW cutoff filter (2680×g until ˜3 mL of solution remained). The centrifuge filtration process was carried out a total of 6 times. The final pH of the solution was adjusted to about 7.4 to about 7.7 using concentrated HCl as necessary.

Preparation of C5-S104. To a vial containing 3.25 mg Fe/mL 5 nm SPIO in tetrahydrofuran (4.0 mL, 13 mg Fe, 0.232 mmol) was added tetrahydrofuran (10 mL) followed by 50% PEI silane in isopropyl alcohol (2.0 mL) and the resulting cloudy solution was sonicated for 2 hours. Isopropanol (4.0 mL) was then added and the solution was sonicated for an additional 16 hours. Concentrated NH₄OH (1.0 mL, 14.8 mmol) was then added and the solution was stirred at room temperature for 4 hours. The solution was then diluted with H₂O (10 mL) and extracted with hexanes (3×10 mL) and etoleic acid (3×10 mL). Any remaining organics in the aqueous layer were removed in vacuo. The resulting homogeneous aqueous solution was passed through a 200 nm followed by a 100 nm syringe filter. The solution was then diluted with H₂O (10 mL total volume) and purified using a 100 kDa MW cutoff filter (2680×g until ˜3 mL of solution remained). The centrifuge filtration process was carried out a total of 6 times. The final pH of the solution was adjusted to about 7.4 to about 7.7 using concentrated HCl as necessary.

Preparation of C5-S105. To a vial containing 2.24 mg Fe/mL 5 nm SPIO in tetrahydrofuran (4.0 mL, 8.96 mg Fe, 0.160 mmol) was added a solution containing trimethylsilyl-glucose-silane (1.19 g, 1.73 mmol) dissolved in tetrahydrofuran (4 mL). Additional tetrahydrofuran (2 mL) was added to the vial. The resulting solution was sonicated for 2 hours. Isopropanol (2.7 mL) was then added and the solution was sonicated for an additional 16 hours. Concentrated NH₄OH (0.854 mL, 9.9 mmol) was then added and the solution was stirred at room temperature for 8 hours. The solution was then diluted with H₂O (10 mL) and extracted with hexanes (3×10 mL) and etoleic acid (3×10 mL). Remaining organics were removed in vacuo. The resulting homogeneous aqueous solution was passed through a 200 nm followed by a 100 nm syringe filter and a 20 nm syringe filter. The solution was then diluted with H₂O (10 mL total volume) and purified using a 100 kDa MW cutoff filter (2680×g until ˜3 mL of solution remained). The centrifuge filtration process was carried out a total of 3 times. Further centrifuge filtration process caused irreversible nanoparticle aggregation.

Physical Characterization of the SPIO Molecules.

Silane-based ligand exchange reactions using S102 and S104 were carried out in the absence of isopropyl alcohol and with only a 2 hour sonication followed by stirring at room temperature with NH₄OH and both processes yield water soluble nanoparticles with similar D_{H} and surface charge.

Toxicology of the SPIO agents: The following toxicology examples describe toxic attributes of several SPIO agents. Cell viability studies using RAW 264.7 mouse macrophage cells and C5-S104 prepared without added isopropyl alcohol show a decrease in cell viability when compared to C5-S104 prepared with isopropyl alcohol. Since water-soluble nanoparticles with similar properties can be prepared in the absence of isopropyl alcohol, it is assumed that the isopropyl alcohol removes agents toxic to inflammatory response cells. Analysis of the centrifuge filtrate for material prepared with and without isopropyl alcohol show the presence of free Fe as evidenced by reaction with potassium ferrocyanate and elemental analysis.

Analytical Data. The analytical data for the core/shell nanoparticles, shown in Table 1, includes the hydrodynamic size, surface charge, as well as the relaxivity values (R1, R2, and R2/R1) of the multiple core/shell particles described herein. Measurement of D_{H}, the surface potential (ζ), (for samples with silane based coatings) are standard analyses performed to determine batch quality and purity.

TABLE 1
Analytical Data for 5 nm Coated SPIO Agents

| | | | R1 | R2 | |
|---|---|---|---|---|---|
| Shell | D_{H} (nm) | ζ (mV) | (mM⁻¹ s⁻¹) | (mM⁻¹ s⁻¹) | R2/R1 |
| C5- | 22.1 ± 2.5 | 17.8 ± 3.1 | 4.5 ± 1.4 | 25 ± 4.2 | 5.5 ± 0.5 |
| S101 | | | | | |
| C5- | 12.2 ± 1.5 | −31.7 ± 3.9 | 15.1 ± 2.0 | 50.8 ± 6.3 | 3.4 ± 0.1 |
| S102 | | | | | |
| C5- | 26.8 ± 8.6 | −17.0 ± 0.5 | 18.35 | 76.26 | 4.16 |
| S103 | | | | | |
| C5- | 13.8 ± 1.4 | 34.0 ± 3.1 | 14.5 | 48.2 | 3.3 |
| S104 | | | | | |
| C5- | 12.7 | −27.0 | | | |
| S105 | | | | | |

^{a}Si/Fe mass ratio

Aggregation. One analytical parameter for measuring nanoparticle aggregation is the hydrodynamic size as measured by dynamic light scattering (DLS) in aqueous solutions. For C5-S101, C5-S102, C5-S103, C5-S104, and C5-S105, D_{H} value greater than about 30 nm is indicative of particle aggregation.

Relaxivity. C5-S101 has a R2/R1 ratio >5 while C5-S102, C5-S103, and C5-S104 have R2/R1 ratios between 3.3 and 4.2. The low cellular uptake of C5-S102 coupled with a R2/R1 ratio of 3.4 allows its use as a positive blood pool agent.

Cellular Uptake

Macrophage Assays. The following assays describe efficient uptake of the SPIO agents of the invention by inflammatory cells, specifically the RAW 264.7 macrophage cell line (ATCC TIB-71).

RAW 264.7 cells are cultured in full media (DMEM+10% FBS) to >80% confluence in all assays. Cells are counted and viability determined by trypan blue assay. SPIOs are sterile-filtered using 100 nm filters and diluted for loading into sterile 1× PBS buffer in a final volume of no more than 5% of the total volume of media in the well (i.e., no more than 100 uL of SPIO solution in 2 mL of full media in single well of a 6-well plate). For all additions to cell culture, the molar amount of Fe is calculated according to stock concentration of the starting SPIO preparation, which is given in mg Fe/mL solution. Most assays use at least 100 micromolar Fe per well of cells.

The SPIO agents were added directly to media to a final concentration of between 10-100 μM. Cells are then cultured under normal conditions for 24 hours to allow for uptake of SPIOs and counted using the trypan blue assay to determine total number of cells and total number of viable cells. The SPIO-treated cells are compared to 1× PBS (sham) treated cells. SPIO agents do not cause significant cytotoxicity if the SPIO preparations are sufficiently clarified and pH adjusted to ˜7 to ˜7.4. Failing to adjust the pH of the SPIO preparation resulted in cytotoxicity.

Flow Cytometry Analysis. RAW264.7 mouse macrophages were treated C5-S104 for 12 hours at 200 uM per well. Samples were fixed in 1% paraformaldehyde before proceeding with flow cytometry. The samples were then subjected to flow cytometry. Acquisition and analysis settings and gates were created to include most of the live cells in acquisition and analysis while eliminating dead cells and cellular debris. Gating for high, medium and low granularity was accomplished by placing analysis gates in the dot plot of forward-angle scatter versus side-angle scatter in regions customarily associated with cells with high, medium, and low granularity values, respectively. Following cell counting, cells were stained to indicate the relative uptake of the SPIOs. A modified version of the Prussian blue stain was used to verify the presence of significant quantities of internalized SPIO agents.

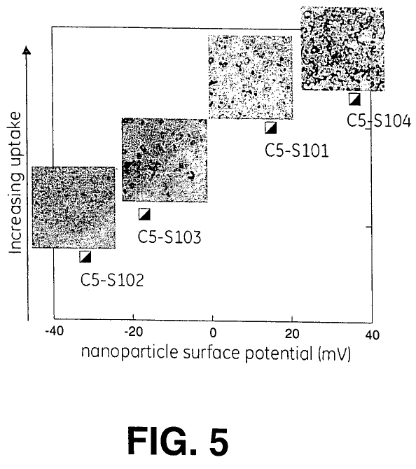

FIG. 5 shows a plot of macrophage uptake as a function of SPIO type. The SPIO core is kept constant at 5 nm and the coating type is varied. All of the SPIOs are approximately the same hydrodynamic size, less than 20 nm.

Animal Studies. The following examples describe two animal models for inflammatory disease, specifically ischemia-reperfusion and MS, along with in vivo application of the SPIO agents to these animal models.

Unilateral Renal Ischemia Reperfusion in SD Rat. Models of renal ischemia-reperfusion in the rat demonstrate that inflammatory cells infiltrate the recovering kidney in a time-specific manner following injury. Macrophage density peaks at approximately 4 days post-injury, as indicated by dense populations of ED-1 positive cells in the outer medulla of the injured kidney. Imaging studies using Sinerem® at a dose of 28 mg Fe/kg body weight in a rat with bilateral I/R injury showed that these particles were visible in the damaged kidneys at day 4 post-injury. Injections are made at day 3 and imaging is performed 24 hours later. The ischemia-reperfusion model, described herein, produces a predictable influx of activated monocytes in the damaged kidney of a Sprague Dawley rat.

Sprague Dawley rats are anesthetized using gas anesthesia (5% isofluorane) and gently placed on a stainless steel heated bed that supplies 2%-3% isofluorane. The abdomen is shaved and cleaned with betadine and alcohol swabs. Eye ointment is placed on the eyes to prevent drying. A lateral incision is then made in the left retroperitoneal area approximately one cm below the rib cage. The left renal pedicle is blunt dissected and a sterile suture then gently placed around the pedicle. Tightening the suture for 60 minutes induces ischemia. In control animals, the suture was not tightened. The abdomen is covered with a warm and sterile moist gauze pad to prevent evaporation and heat loss during surgery.

Following ischemia or sham time, the suture is gently removed. The incision is sutured in layers using 4-0 silk sutures. Animals are allowed to recover for at least 3 days. The animals are then anesthetized and injected via tail vein with SPIO agent at dose of 1 mg Fe/kg body weight.

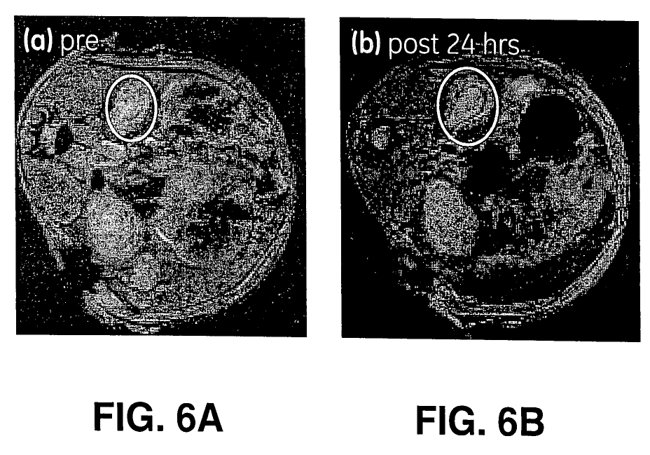

FIG. 6B shows an in vivo MR image of an injured rat 3 days post surgery. The kidneys show no difference in the pre-injection images. These In vivo images are obtained one day post injection of C5-S104 at a dose of 1 mg Fe/kg bw. T2 weighted images are obtained using a spin echo sequence (TR=1400 ms, TE=30 ms, FOV=8, slice thickness=1.5 mm). A signal loss on a T2 weighted image (yellow circle) is observed in the outer medulla of the injured kidney while no change in signal is observed in the normal kidney (as seen in lower quadrant of FIG. 6B). This signal loss is caused by the presence of the SPIO agent in the inflamed tissue of the injured kidney.

Tissues are collected from animals within 1 hour of imaging session without perfusion. Tissues are then fixed in formalin for at least 24 hours before processing and paraffin-embedding for sectioning. Sections are made at 6 um thickness and stained by standard hematoxylin and eosin protocols (H&E). Sections are also stained for iron following standard Prussian blue staining protocol. H&E staining indicate severe damage to left kidney and a significant number of infiltrating cells. Prussian blue staining further indicated the presence of iron-positive species.

MS Model. A MS model was developed which utilizes a unique form of experimental autoimmune encephalomyelitis (EAE) in the Dark Agouti rat. In the model, macrophage-containing lesions form in the white matter of the brain. Using the model SPIO uptake in the brain may be observed over the progression of the disease. The EAE model has been improved by (1) eliminating several compounds from the injection mixture that both created a more debilitating and lethal form of EAE in the rat and (2) removing an additive that created a severe allergic response in most animals.

Induction of EAE is known to be both more reliable and less severe in the Dark Agouti (DA) rat relative to other strains. DA rats are more susceptible to EAE due to known immunological differences between DA and Lewis rats and do not require the use of adjuvants to induce EAE. Because adjuvants can cause inflammatory and immune responses in rats without using the desired immunogen, it is desirable to study this MS disease model in a strain that does not require use of adjuvant to stimulate EAE.

Animals are anesthetized (1.5% isofluorane) and receive an intradermal injection of rat spinal cord homogenate (from Sprague-Dawley rats) in the right hind footpad to induce EAE. The injection site is treated with a local pain reliever (Mobisyl cream (active ingredient trolamine salicytate) administered to foot near injection site, twice per day for 2 days).

Animals typically demonstrate clinical symptoms of EAE between days 8-10 post-injection of homogenate, indicated by weight loss >10 g in one day and loss of tail tonus. Within 2 days of the first symptoms, animals experience complete hind limb paralysis and are given fluids (2 mL 0.9% saline) subcutaneous twice per day. At clinical score of 3, animals are intravenously injected with C5-S104 via tail vein at dose of 1-2 mg Fe/kg body weight. All injections are done while animal is under anesthesia (1.5% isofluorane).

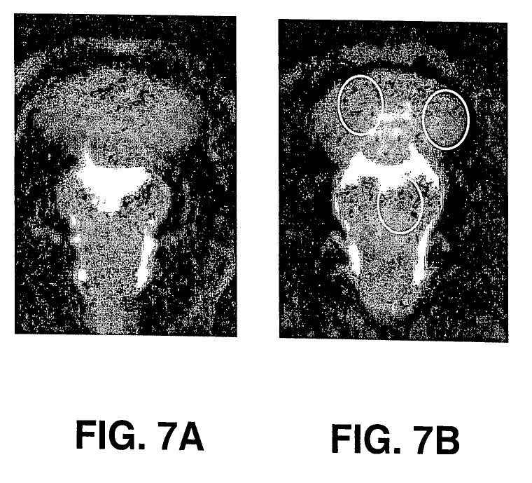

In vivo magnetic resonance imaging is performed using a GE Signa 1.5 scanner. A T2 weighted fast spin echo sequence (TR=4500 ms, TE=63 ms, FOV=5, slice thickness=1.5 mm) is used. FIG. 7A shows an image of the brain of a normal DA rat. FIG. 7B shows an image of an EAE rat 24 hours after injection of C5-S104 agent at a dose of 1 mg Fe/kg bw. Areas of low signal intensity are observed within the medulla and the brain stem of the injected rat. These areas are indicative of SPIO accumulation within the lesions.

EAE rats and controls are sacrificed immediately following imaging for harvest of tissue. Animals are perfused at physiological pressure twice with 60 mL saline and twice with 60 mL formalin. Brains are then extracted and formalin-fixed for an additional 24 hours before processing and paraffin-embedding. Histological study of the brain from the injected EAE rat indicates iron uptake in focal region of the medulla, presumably from SPIO (FIG. XX). Higher magnification of the lesion clearly shows the localization of the SPIO within a host of cells.

Atherosclerosis. In this atherosclerosis model, mice develop focal, occlusive and macrophage-dense plaques within 14 days of injury to the carotid artery. ApoE −/− mice on a C57/BL6 background were anesthetized by intraperitoneal injection of xylazine/ketamine before surgery. The left common carotid was then ligated using 5-0 silk, and the animals were allowed to recover. Animals were then kept on western diet (21% fat, 0.15% cholesterol) for 14 days following the surgery. Fourteen days post-ligation, animals (n=6) were injected via tail vein with 0.2 mg Fe/kg body weight of CS-104. Animals were sacrificed at 24 hours (n=3) and 48 hours (n=3) post-injection of CS-104; the left and right carotid arteries were collected and fixed in formalin.

Tissues were then embedded in paraffin and sectioned (5 microns per slice). Sections were then stained for iron and for cellular infiltrate. Sections containing the occlusive plaque showed dense cellular and lipid infiltrates in the left common carotid and also showed high density of iron-positive cells. Sections from right carotid arteries did not demonstrate plaques or iron-positive cells.

### EQUIVALENTS

The invention may be embodied in other specific forms without departing from the spirit or essential characteristics thereof. The foregoing embodiments are thereof to be considered in all respects illustrative rather than limiting on the invention described herein. Scope of the invention is thus indicated by the appended claims rather than by the foregoing description, and all changes that come within the meaning and range of equivalency of the claims are therefore intended to be embraced therein.

1. A method of imaging an inflammatory condition in a mammal comprising introducing into the mammal a positively charged SPIO agent including a superparamagnetic core and a cationic coating into inflammatory cells in vivo or ex vivo, permitting the inflammatory cells to migrate to inflamed tissue, imaging the inflamed tissue using magnetic resonance, and, optionally, managing the inflammatory condition.

2. The method of claim 1, wherein the mammal is a human.

3. The method of claim 1, further comprising treating the mammal to decrease inflammation before, after, or before and after imaging the inflammatory condition, and using the results to manage the inflammatory condition.

4. The method of claim 1, wherein the core is superparamagnetic.

5. The method of claim 1, wherein the core comprises divalent metal ions.

6. The method of claim 5, wherein the divalent metal irons include iron, manganese, nickel, cobalt, magnesium, or a combination thereof.

7. The method of claim 1, wherein the size of the core is about 2 nm to about 200 nm.

8. The method of claim 7, wherein the size of core is about 2 nm to about 100 nm.

9. The method of claim 7, wherein the size of core is about 5 nm to about 9 nm.

10. The method of claim 7, wherein the size of core is about 9 nm.

11. The method of claim 7, wherein the size of core is about 7 nm.

12. The method of claim 7, wherein the size of core is about 5 nm.

13. The method of claim 1, wherein the agent is substantially coated with PEG, PEI, or combinations thereof.

14. The method of claim 13, wherein the PEG coating comprises: PEG-silane, PEG-dendron; PEG-dendron-silane, or combinations thereof.

15. The method of claim 13, wherein the coating comprises PEI.

16. The method of claim 13, wherein the coating comprises PEG with a molecular weight between about 350 Da to about 5000 Da.

17. The method of claim 13, wherein the coating comprises PEG with a molecular weight between about 550 Da to about 1000 Da.

18. The method of claim 13, wherein the coating and shell comprises: 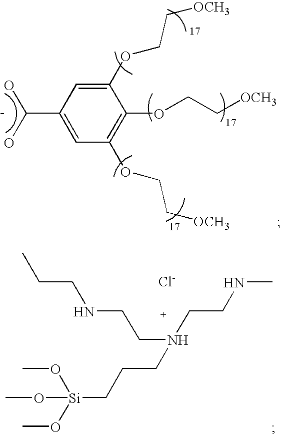
or combinations thereof.

19. The method of claim 1, wherein the D_{H} of the core and coating is about 3 nm to about 50 nm.

20. The method of claim 17, wherein the D_{H} of the core and coating is about 17 nm.

21. The method of claim 1, wherein the zeta potential of the agent is greater than about 0 and less than about +60 mV.

22. The method of claim 1, wherein the zeta potential of the agent is about +20 mV to about +40 mV.

23. The method of claim 1, wherein the zeta potential of the agent is about +40 mV.

24. The method of claim 1, wherein the agent is less than about 15% polydispersed.

25. The method of claim 1, wherein the R1 relaxivity of the agent is greater than about 4 mM⁻¹s⁻¹.

26. The method of claim 1, wherein the R2 relaxivity of the agent is greater than about 20 mM⁻¹s⁻¹.

27. The method of claim 1, wherein the R2/R1 ratio of the agent is greater than about 2.

28. The method of claim 1, wherein the agent is dispersed in a biocompatible solution with a pH of about 6 to about 8.

29. The method of claim 1, wherein the agent is dispersed in a biocompatible solution with a pH of about 7 to about 7.5.

30. The method of claim 1, wherein the agent is dispersed in a biocompatible solution with a pH of about 7.4.

31. The method of claim 1, wherein the blood half-life of the agent is about 30 minutes to about 48 hours.

32. The method of claim 1, wherein the blood half-life of the agent is about 30 minutes to about 2 hours.

33. The method of claim 1, wherein the introducing step comprises administering the agent topically, intravascularly, intramuscularly, or interstitially.

34. The method of claim 2, wherein the about 0.1 mg Fe/kg to about 50 mg Fe/kg of agent is administered to the mammal.

35. The method of claim 2, wherein the about 0.2 mg Fe/kg to about 2.5 mg Fe/kg of agent is administered to the mammal.

36. The method of claim 1, wherein the condition is associated with macrophage accumulation.

37. The method of claim 1, wherein the condition is an autoimmune condition, a vascular condition, a neurological condition, or a combination thereof.
